(19) United States
(12) Patent Application Publication
(10) Pub. No.: US 2022/0211330 A1
(43) Pub. Date: Jul. 7, 2022

(54) METHOD AND DEVICE FOR GENERATING HEART MODEL REFLECTING ACTION POTENTIAL DURATION RESTITUTION

(71) Applicant: INDUSTRY-ACCADEMI CCOOPERATION FOUNDATION YONSEI UNIVERSITY, Seoul (KR)

(72) Inventors: Hui Nam PAK, Seoul (KR); Byoung Hyun LIM, Seoul (KR)

(21) Appl. No.: 17/604,482

(22) PCT Filed: May 7, 2020
(86) PCT No.: PCT/KR2020/005989

§ 371 Date: Oct. 18, 2021

(30) Foreign Application Priority Data
May 29, 2019 (KR) 10-2019-0063369

(51) Int. Cl. A61B 5/00; A61B 5/318; A61B 5/361; G06F 30/20; G09B 23/28; G16H 50/50

(52) U.S. Cl. CPC A61B 5/7275; A61B 5/318; A61B 5/361; G16H 50/50; G06F 30/20; G09B 23/28; A61B 5/7264

(57) ABSTRACT

A method for generating a heart model reflecting action potential duration restitution is provided, which can visually output the maximum slope of the correlation between a relaxation period and an action potential duration at every point included in a three-dimensional heart model.

### TECHNICAL FIELD

The present invention relates to a method and device for generating a heart model reflecting an action potential duration restitution. More specifically, the present invention relates to a method and device for generating a heart model reflecting an action potential duration restitution, which can visually output the maximum slope of the correlation between a relaxation period and an action potential duration at every point included in a three-dimensional heart model.

### BACKGROUND ART

Arrhythmia means a symptom of abnormally fast, slow, or irregular heartbeats, which occurs as the heart does not produce electrical stimulation well or the stimulation is not transferred properly due to occurrence of atrial fibrillation, and thus the heart does not continue to contract regularly, and it provides a cause of sudden death or stroke.

As a method of treating the arrhythmias, there is a surgical therapy, such as a radiofrequency catheter ablation procedure, that can block electrical conduction of the heart by cauterizing heart tissues to prevent the arrhythmias. However, there is a problem in that it is difficult to grasp in advance at what intensity the ablation procedure should be performed at which part of the heart to derive an optimal effect.

The problem of the radiofrequency catheter ablation procedure can be solved on condition that a point where atrial fibrillation occurs and a point where the atrial fibrillation is highly likely to occur can be accurately detected before the radiofrequency catheter ablation procedure, since atrial fibrillation that occurs can be removed and atrial fibrillation that will occur in the future can be prevented at the same time by performing the radiofrequency catheter ablation procedure at these points.

Meanwhile, in the prior art, a time/frequency analysis method using an electrocardiography (ECG) signal has been developed in relation to the points where atrial fibrillation occurs. However, as the electrocardiogram signal itself is exposed to noise and has a limited data length and non-stationary, it is difficult to accurately detect the points where the atrial fibrillation occurs, and the cost consumed for the time/frequency analysis method itself is considerably high, and furthermore, there is also a problem in that the points where the atrial fibrillation is highly likely to occur are difficult to detect.

Accordingly, it is required to develop a new technique that can accurately detect the points where atrial fibrillation occurs and the points where atrial fibrillation is highly likely to occur before the radiofrequency catheter ablation procedure at an affordable cost. The present invention relates to this technique.

### DISCLOSURE OF INVENTION

### Technical Problem

Therefore, the present invention has been made in view of the above problems, and it is an object of the present invention to provide a method and device capable of accurately detecting a point where atrial fibrillation occurs and a point where atrial fibrillation is highly likely to occur before a radiofrequency catheter ablation procedure.

Another object of the present invention is to provide a method and device capable of minimizing the economic burden of a patient by detecting a point where atrial fibrillation occurs and a point where atrial fibrillation is highly likely to occur at an affordable cost.

Technical problems of the present invention are not limited to the technical problems mentioned above, and other technical problems not mentioned will be clearly understood by those skilled in the art from the following description.

### Technical Solution

To accomplish the above objects, according to one aspect of the present invention, there is provided a heart model generation method reflecting action potential duration restitution, the method comprising the steps of: (a) loading a heart model including N (N is a natural number equal to or greater than 1) coordinates, and time-specific voltage data including voltage values measured at N coordinates included in the heart model at first predetermined time intervals; (b) calculating a relaxation period that is a time period from a point APD90 showing a voltage value dropped 90% from the highest point of the voltage value included in a first predetermined time interval to an electrically stimulated point included in a next first predetermined time interval at specific coordinates included in the heart model using the loaded time-specific voltage data; (c) calculating an action potential duration that is a time period from an electrically stimulated point included in the next first predetermined time interval to a point APD90 showing a voltage value dropped 90% from the highest point of the voltage value included in the next first predetermined time interval at specific coordinates included in the heart model using the loaded time-specific voltage data; (d) calculating a correlation between the relaxation period and the action potential duration at specific coordinates included in the calculated heart model, and calculating a maximum slope using the calculated correlation; and (e) visually outputting the calculated maximum slope by reflecting the maximum slope to specific coordinates included in the heart model.

According to an embodiment, the heart model may be a three-dimensional atrium model generated for each patient.

According to an embodiment, the N coordinates may be 450,000 coordinates.

According to an embodiment, the first predetermined time interval may be any one among 1 ms, 2 ms, and 3 ms.

According to an embodiment, the correlation between the relaxation period and the action potential duration at step (d) may be calculated through a correlation calculation formula shown below.

y(action potential duration)=y₀−A₁(1−e^{-relaxation period/τ1}) Correlation calculation formula:

(Here, yₒ and A₁ are free-fitting variables, and τ₁ is a time constant.)

According to an embodiment, the maximum slope may be calculated by differentiating the correlation calculation formula with respect to the relaxation duration.

According to an embodiment, the heart model generation method may further comprise, after step (e), the step of (f) repeatedly performing steps (b) to (e) for all the N coordinates included in the heart model except the specific coordinates.

According to an embodiment, the heart model generation method may further comprise, after step (f), the step of (g) applying an interpolation method to the maximum slope calculated for the N coordinates included in the heart model, and visually outputting the maximum slope, for remaining areas of the heart model except the N coordinates included in the heart model.

According to an embodiment, a range of a magnitude of the calculated maximum slope may be 0.3 to 2.3, and a visual output of step (e) may be outputting the maximum slope in a different color according to the magnitude of the calculated maximum slope.

According to another aspect of the present invention, there is provided a heart model generation device reflecting action potential duration restitution, the device comprising: one or more processors; a network interface; a memory for loading a computer program executed by the processors; and a storage for storing large-scale network data and the computer program, wherein the computer program executes, by the one or more processors 10, (a) an operation of loading a heart model including N (N is a natural number equal to or greater than 1) coordinates, and time-specific voltage data including voltage values measured at N coordinates included in the heart model at first predetermined time intervals, (b) an operation of calculating a relaxation period that is a time period from a point APD90 showing a voltage value dropped 90% from the highest point of the voltage value included in a first predetermined time interval to an electrically stimulated point included in a next first predetermined time interval at specific coordinates included in the heart model using the loaded time-specific voltage data, (c) an operation of calculating an action potential duration that is a time period from an electrically stimulated point included in the next first predetermined time interval to a point APD90 showing a voltage value dropped 90% from the highest point of the voltage value included in the next first predetermined time interval at specific coordinates included in the heart model using the loaded time-specific voltage data, (d) an operation of calculating a correlation between the relaxation period and the action potential duration at specific coordinates included in the calculated heart model, and calculating a maximum slope using the calculated correlation, and (e) an operation of visually outputting the calculated maximum slope by reflecting the maximum slope to specific coordinates included in the heart model.

According to still another aspect of the present invention, there is provided a computer program stored in a medium to execute, combination with a computing device, the steps of: (a) loading a heart model including N (N is a natural number equal to or greater than 1) coordinates, and time-specific voltage data including voltage values measured at N coordinates included in the heart model at first predetermined time intervals; (b) calculating a relaxation period that is a time period from a point APD90 showing a voltage value dropped 90% from the highest point of the voltage value included in a first predetermined time interval to an electrically stimulated point included in a next first predetermined time interval at specific coordinates included in the heart model using the loaded time-specific voltage data; (c) calculating an action potential duration that is a time period from an electrically stimulated point included in the next first predetermined time interval to a point APD90 showing a voltage value dropped 90% from the highest point of the voltage value included in the next first predetermined time interval at specific coordinates included in the heart model using the loaded time-specific voltage data; (d) calculating a correlation between the relaxation period and the action potential duration at specific coordinates included in the calculated heart model, and calculating a maximum slope using the calculated correlation; and (e) visually outputting the calculated maximum slope by reflecting the maximum slope to specific coordinates included in the heart model.

### Advantageous Effects

According to the present invention as described above, since the slope with respect to the correlation between the relaxation period and the action potential duration is visually output to the heart model in real-time, there is an effect in that a user may accurately detect a point where atrial fibrillation occurs and a point where atrial fibrillation is highly likely to occur before a radiofrequency catheter ablation procedure while confirming a finally output heart model in real-time.

In addition, since the time-specific voltage data used in generating the finally output heart model is a result data of a test generally performed for patients of arrhythmia, and the cost is not high, there is an effect of minimizing the economic burdens of the patients.

The effects of the present invention are not limited to the effects mentioned above, and other effects not mentioned will be clearly understood by those skilled in the art from the following description.

### BRIEF DESCRIPTION OF THE DRAWINGS

FIG. 1 is a view showing the overall configuration included in a heart model generation device reflecting action potential duration restitution according to a first embodiment of the present invention.

FIG. 2 is a flowchart illustrating the representative steps of a heart model generation method reflecting action potential duration restitution according to a second embodiment of the present invention.

FIG. 3 is a view exemplarily showing a heart model including N coordinates.

FIG. 4 is a view exemplarily showing time-specific voltage data including voltage values measured at every first predetermined time intervals at N coordinates included in the heart model.

FIG. 5 is an enlarged view showing part of a voltage value among voltage values measured at every first predetermined time intervals at any specific coordinates among first to N-th coordinates shown in FIG. 4.

FIG. 6 is a view additionally showing a relaxation period in the view shown in FIG. 5.

FIG. 7 is a view additionally showing the action potential duration in the view shown in FIG. 6.

FIG. 8 is a view showing the correlation between a relaxation period and an action potential duration during measurement at specific coordinates as an exemplary graph through a correlation calculation formula.

FIG. 9 is a view additionally showing the maximum slope among a plurality of slopes in the view shown in FIG. 8.

FIG. 10 is a view showing the maximum slope at specific coordinates in color in the heart model shown in FIG. 3.

FIG. 11 is a flowchart illustrating the steps performed after step S250 in the flowchart shown in FIG. 2.

FIG. 12 is a view showing the maximum slope in the entire area in color by applying an interpolation method to the heart model shown in FIG. 10.

FIG. 13 is a view showing the maximum slope at corresponding coordinates, numerically output when a user selects specific coordinates of a heart model through a mouse.

FIG. 14 is a view showing a heart model output together with the stimulation cycles of an electrical signal.

### BEST MODE FOR CARRYING OUT THE INVENTION

Hereinafter, preferred embodiments of the present invention will be described in detail with reference to the accompanying drawings. Advantages and features of the present invention, and a method for achieving them will become apparent with reference to the embodiments described below in detail, together with the accompanying drawings. However, the present invention is not limited to the embodiments described below, and may be implemented in various different forms. These embodiments are provided only to make the disclosure of the present invention complete and to fully inform the scope of the present invention to those skilled in the art, and the present invention is only defined by the scope of the claims. Like reference numerals refer to like elements throughout the specification.

Unless otherwise defined, all terms (including technical and scientific terms) used in this specification may be used as a meaning commonly understood by those skilled in the art. In addition, terms defined in a generally used dictionary are not to be interpreted ideally or excessively unless clearly defined in particular. The terms used in this specification are for describing the embodiments and are not intended to limit the present invention. In this specification, singular forms also include plural forms unless specially stated otherwise in the phrase.

The terms “comprises” and/or “comprising” used in this specification mean that a mentioned component, step, operation, and/or element does not exclude presence or addition of one or more other components, steps, operations, and/or elements.

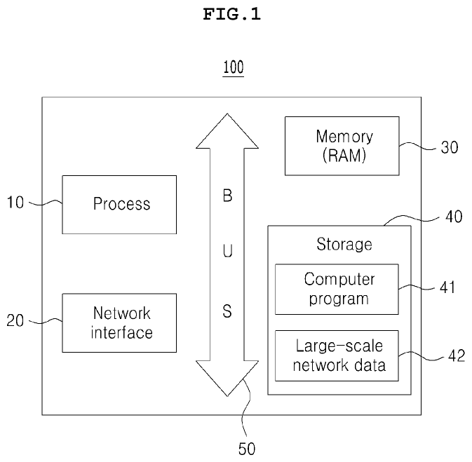

FIG. 1 is a view showing the overall configuration included in a heart model generation device 100 reflecting action potential duration restitution according to a first embodiment of the present invention.

However, this is only a preferred embodiment for achieving the object of the present invention, and some components may be added or deleted as needed, and of course, a function performed by one component may be performed by another component.

A heart model generation device 100 reflecting action potential duration restitution according to a first embodiment of the present invention includes a processor 10, a network interface 20, a memory 30, a storage 40, and a data bus 50 for connecting them.

The processor 10 controls the overall operation of each component. The processor 10 may be any one among a central processing unit (CPU), a microprocessor unit (MPU), a microcontroller unit (MCU), and a type of processor widely known in the art. In addition, the processor 10 may perform an operation with regard to at least one application or program for performing a heart model generation method reflecting action potential duration restitution according to a second embodiment of the present invention.

The network interface 20 supports wired/wireless Internet communication of the heart model generation device 100 reflecting action potential duration restitution according to a first embodiment of the present invention, and may support other known communication methods. Accordingly, the network interface 20 may be configured to include a communication module according thereto.

The memory 30 may store various data, commands and/or information, and may load one or more computer programs 41 from the storage 40 to perform the heart model generation method reflecting action potential duration restitution according to a second embodiment of the present invention. Although RAM is shown in FIG. 1 as the memory 30, it goes without saying that various storage media may be used as the memory 30.

The storage 40 may permanently store one or more computer programs 41 and large-scale network data 42. The storage 40 may be any one among non-volatile memory such as read only memory (ROM), erasable programmable ROM (EPROM), electrically erasable programmable ROM (EEPROM), flash memory and the like, a hard disk, a removable disk, and computer-readable recording media of an arbitrary form widely known in the technical field to which the present invention belongs.

The computer program 41 may be loaded on the memory 30 and execute, by one or more processors 10, (a) an operation of loading a heart model including N (N is a natural number equal to or greater than 1) coordinates, and time-specific voltage data including voltage values measured at N coordinates included in the heart model at first predetermined time intervals, (b) an operation of calculating a relaxation period that is a time period from a point APD90 showing a voltage value dropped 90% from the highest point of the voltage value included in a first predetermined time interval to an electrically stimulated point included in a next first predetermined time interval at specific coordinates included in the heart model using the loaded time-specific voltage data, (c) an operation of calculating an action potential duration that is a time period from an electrically stimulated point included in the next first predetermined time interval to a point showing a voltage value dropped 90% from the highest point of the voltage value included in the next first predetermined time interval at specific coordinates included in the heart model using the loaded time-specific voltage data, (d) an operation of calculating a correlation between the relaxation period and the action potential duration at specific coordinates included in the calculated heart model, and calculating a maximum slope using the calculated correlation, and (e) an operation of visually outputting the calculated maximum slope by reflecting the maximum slope to specific coordinates included in the heart model.

The operations performed by the computer program 41 briefly mentioned so far may be regarded as a function of the computer program 41, and more detailed description will be provided below in the description of a heart model generation method reflecting action potential duration restitution according to a second embodiment of the present invention.

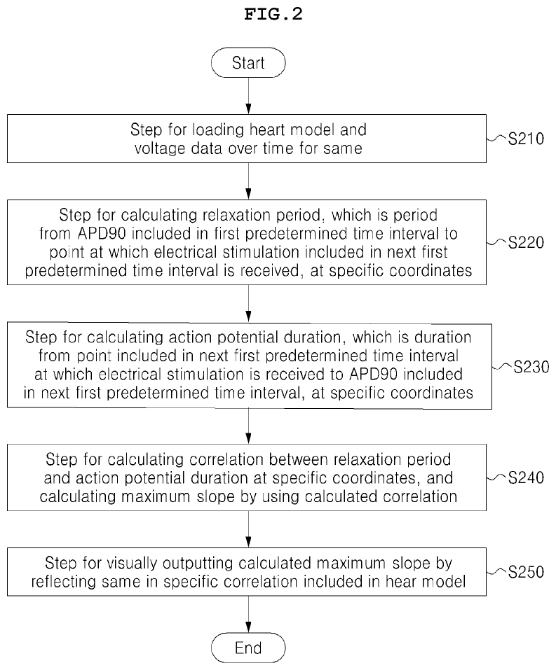

Hereinafter, a heart model generation method reflecting action potential duration restitution according to a second embodiment of the present invention will be described with reference to FIGS. 2 to 14.

FIG. 2 is a flowchart illustrating the representative steps of a heart model generation method reflecting action potential duration restitution according to a second embodiment of the present invention.

This is only a preferred embodiment in achieving the object of the present invention, and some steps may be added or deleted as needed, and furthermore, any one step may be included in another step.

Meanwhile, it is assumed that all steps are performed by the heart model generation device 100 reflecting action potential duration restitution according to a first embodiment of the present invention.

First, the heart model generation device 100 loads a heart model including N (N is a natural number equal to or greater than 1) coordinates, and time-specific voltage data including voltage values measured at N coordinates included in the heart model at first predetermined time intervals (S210).

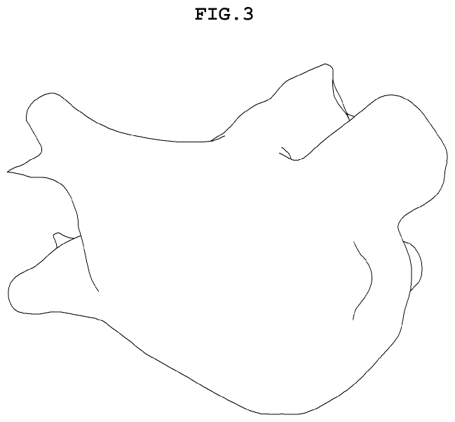

Here, a heart model including N coordinates is exemplarily shown in FIG. 3. Referring to FIG. 3, the heart model may be a three-dimensional atrium model generated for each patient, but it is not necessarily limited thereto, and in some cases, a two-dimensional atrium model may be used. However, since a real heart of a patient has a three-dimensional shape, and a point where atrial fibrillation occurs and a point where atrial fibrillation is highly likely to occur may exist in an area that cannot be expressed two-dimensionally, it will be preferable to use a three-dimensional atrium model.

Meanwhile, although FIG. 3 does not separately show N coordinates that are difficult to visually identify, the N coordinates may be coordinates of specific points in the heart model.

More specifically, although N is a natural number greater than or equal to 1, as the object of the present invention is to detect a point where atrial fibrillation occurs and a point where atrial fibrillation is highly likely to occur from all the points included in the heart model, it is preferable to improve the accuracy by setting N to a high number. For example, N may be a number between 250,000 and 650,000. However, although the calculation speed may increase when N is small, the accuracy may be lowered, and although the accuracy may be improved when N is large, the calculation speed may decrease. Therefore, it will be most preferable to set N to 450,000 in consideration of both the calculation speed and accuracy, and this can be freely set by a designer of the heart model generation device 100 reflecting action potential duration restitution according to a first embodiment of the present invention or a user, such as a doctor, using the device.

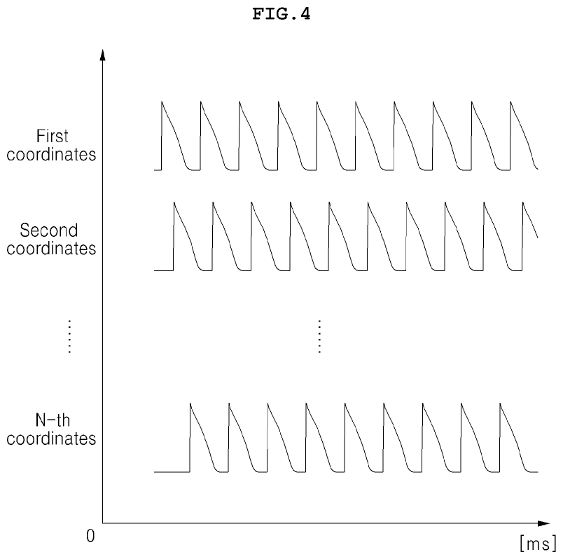

FIG. 4 is a view exemplarily showing time-specific voltage data including voltage values measured at every first predetermined time intervals at N coordinates included in the heart model.

Referring to FIG. 4, it can be confirmed that time-specific voltage data includes all the voltage values measured for all the N coordinates described above. Otherwise, the number of coordinates included in the heart model needs to be synchronized with the number of coordinates where the voltage value included in the time-specific voltage data is measured.

For example, when N coordinates included in the heart model are 450,000 coordinates and the measured voltage values relate to 500,000 coordinates, synchronization for matching the voltage values to 450,000 coordinates will be needed.

However, when the heart model and the time-specific voltage data are simultaneously or sequentially generated through the same device or the same program, since the time-specific voltage data will be generated by measuring voltage values for the N coordinates included in the generated heart model, separate synchronization will not be needed.

The first predetermined time interval may be set in consideration of the periodicity of the voltage value, and the voltage value measured from the heart has a property of repeating with a constant cycle, and this is also exemplarily shown in FIG. 4. Therefore, the first predetermined time interval is preferably set by reflecting the cycle of the voltage value, and it is preferable to set any one among 1 ms, 2 ms, and 3 ms as the first predetermined time interval. In FIG. 4, it can be confirmed that the voltage value is measured using 1 ms as the first predetermined time interval, and the description will be continued based on this.

Meanwhile, although step S210 has been described above based on loading the heart model and the time-specific voltage data, here, the loading corresponds to a case where the heart model and the time-specific voltage data are previously stored in the heart model generation device 100 reflecting action potential duration restitution according to a first embodiment of the present invention, and when the heart model and the time-specific voltage data are received through an external device, the loading may be regarded as an input.

When the heart model and the time-specific voltage data are loaded, an operation is performed to calculate a relaxation period that is a time period from a point APD90 showing a voltage value dropped 90% from the highest point of the voltage value included in a first predetermined time interval to an electrically stimulated point included in a next first predetermined time interval at specific coordinates included in the heart model using the loaded time-specific voltage data (S220).

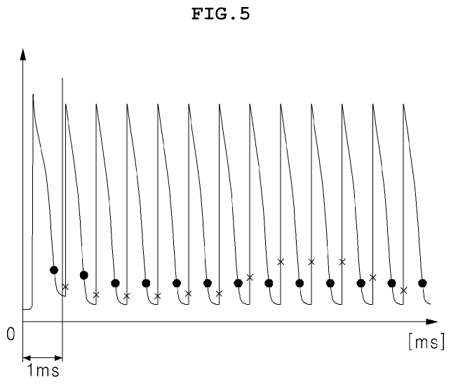

FIG. 5 is an enlarged view showing part of a voltage value among voltage values measured at every first predetermined time intervals at any specific coordinates among first to N-th coordinates shown in FIG. 4, and the first predetermined time interval is 1 ms.

Referring to FIG. 5, it can be confirmed that the voltage values are repeated in a relatively similar tendency at a cycle of 1 ms, which is the first predetermined time interval, and it can be confirmed that mark O and mark X are shown at the voltage values in the first predetermined time interval. Here, the points marked with O are APD90, which are points showing a voltage value dropped 90% from the highest point of the voltage value, and the points marked with X are beginning points of depolarization or repolarization, which are electrically stimulated points described below.

Referring to the voltage value within the first predetermined time interval that begins first, it can be confirmed that the voltage value indicates the highest point at the point around the middle, and since APD90 is a point showing a voltage value dropped 90% from the highest point of the voltage value, it should to be a point after the highest point of the voltage value.

Meanwhile, it also needs to detect an electrically stimulated point, as well as APD90 described above, in order to calculate a relaxation period, and here, detection of the electrically stimulated point is based on a first predetermined time interval next to a first predetermined time interval including the APD90. For example, when it is assumed that a first predetermined time interval starting first among the first predetermined time intervals shown in FIG. 5 is an A-th predetermined time and a next first predetermined time interval is a B-th predetermined time, an electrically stimulated point for calculating a relaxation period for APD90 detected within the A-th predetermined time is a point included in the B-th predetermined time.

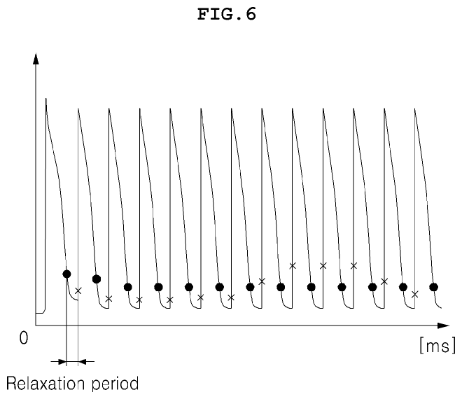

FIG. 6 is a view additionally showing a relaxation period in the view shown in FIG. 5. It can be confirmed that the relaxation period is a period between the APD90 and the electrically stimulated point, more specifically, a period between the APD90 included in the first predetermined time interval and the electrically stimulated point included in the next first predetermined time interval.

Now, return to the description of FIG. 2 again.

When the relaxation period is calculated, an operation is performed to calculate an action potential duration that is a time period from an electrically stimulated point included in the next first predetermined time interval to a point APD90 showing a voltage value dropped 90% from the highest point of the voltage value included in the next first predetermined time interval at specific coordinates included in the heart model using the loaded time-specific voltage data (S230).

Here, since the electrically stimulated point included in the next first predetermined time interval is the same as the electrically stimulated point included in the next first predetermined time interval mentioned in the description of step S220, a detailed description will be omitted to prevent duplicate description.

Meanwhile, although description on the APD90, which is a point showing a voltage value dropped 90% from the highest point of the voltage value included in the next first predetermined time interval, is also basically the same as the APD90 included in the first predetermined time interval mentioned above in the description of step S220, the difference from step S220 is that the APD90 is not a point included in the first predetermined time interval, but a point included in the next first predetermined time interval. For example, when the APD90 in step S220 is a point included in the A-th predetermined time interval, the APD90 in step S230 is a point included in the B-th predetermined time interval.

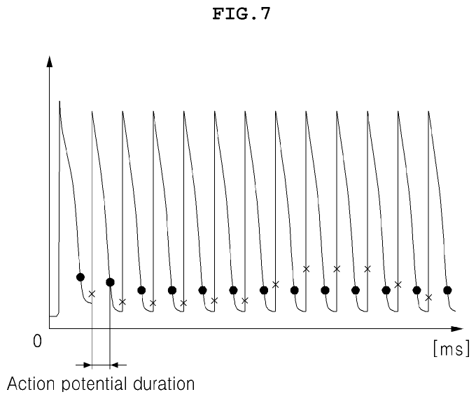

FIG. 7 is a view additionally showing the action potential duration in the view shown in FIG. 6, and it can be confirmed that the action potential duration is the period between an electrically stimulated point and APD90, more specifically, a period between an electrically stimulated point included in the next first predetermined time interval of the first predetermined time interval and the APD90 included in the first predetermined time interval.

Summarizing steps S220 and S230 described above, the end point of the calculated relaxation period becomes the start point of the calculated action potential duration, and the relation between the relaxation period and the action potential duration will be continuously maintained even after the next first predetermined time interval of the first predetermined time interval. That is, the relation of relaxation period—action potential duration—relaxation period—action potential duration—relaxation period—action potential duration . . . will be maintained based on specific coordinates, and accordingly, step S235 in which steps S220 and S230 are repeatedly performed for all measurement times may be further performed after step S230.

In addition, although steps S220 and S230 have been described separately for convenience of explanation, steps S220, S230, and S235 may be simultaneously performed through parallel processing, and in this case, the calculation speed may be dramatically improved.

When the relaxation period and the action transition duration are calculated, the correlation between the relaxation period and the action potential duration at specific coordinates included in the generated heart model is calculated, and then the maximum slope is calculated using the calculated correlation (S240).

Here, the correlation between the relaxation period and the action potential duration at specific coordinates may be calculated through the correlation calculation formula shown below.

y(action potential duration)=y₀−A₁(1−e^{-relaxation period/τ1}) Correlation calculation formula:

Here, yₒ and A₁ are free-fitting variables, τ₁ is a time constant, yₒ may be set to 50 initially, the relaxation period may be set to 10, and τ1 may be set to 30, and it is possible to freely set within a range having a minimum value of −50, −10, and −30 and a maximum value of 1000, 1000, and 1000, respectively.

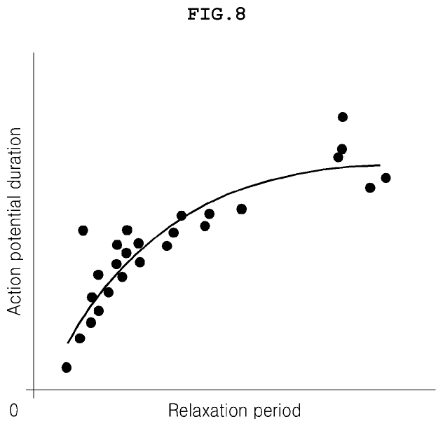

FIG. 8 is a view showing the correlation between a relaxation period and an action potential duration at specific coordinates as an exemplary graph through a correlation calculation formula, and since the correlation is a kind of function as is confirmed with reference to the correlation calculation formula itself and FIG. 8, the slope can be calculated by performing differentiation on the relaxation period.

(A₁/τ₁)·e^{(-relaxation period/τ1)} Slope:

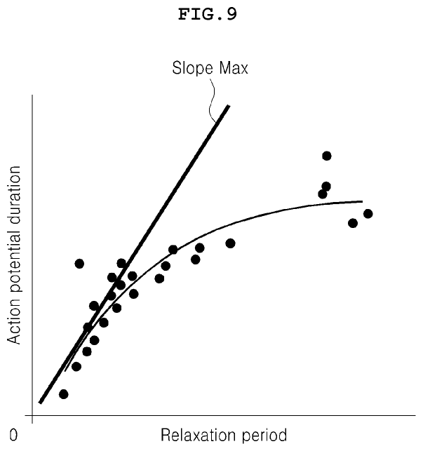

Meanwhile, since the slope to be calculated at step S240 is the maximum slope, when only one relaxation period and one action potential duration are calculated at specific coordinates, the slope with respect to the correlation between the relaxation period and the action potential duration will be the maximum slope. However, since the relaxation period and the action potential duration can be calculated for all measurement times at specific coordinates as step S235 is performed before, in this case, there will be a plurality of calculated slopes, and the largest slope among them may be calculated as the maximum slope, and FIG. 8 is also shown based on this, and the maximum slope among the plurality of slopes is separately shown in FIG. 9.

When the maximum slope is calculated, the calculated maximum slope is reflected to specific coordinates included in the heart model and visually output (S250).

Here, the visual output may be implemented through various methods, and the maximum slope may be output in a different color at corresponding coordinates according to the magnitude of the calculated maximum slope, or the numeric value of the maximum slope, for example, the numeric value of the magnitude of the corresponding maximum slope, may be directly output within a range between 0.3 and 2.3.

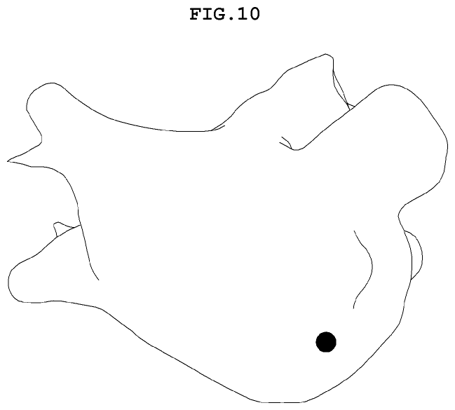

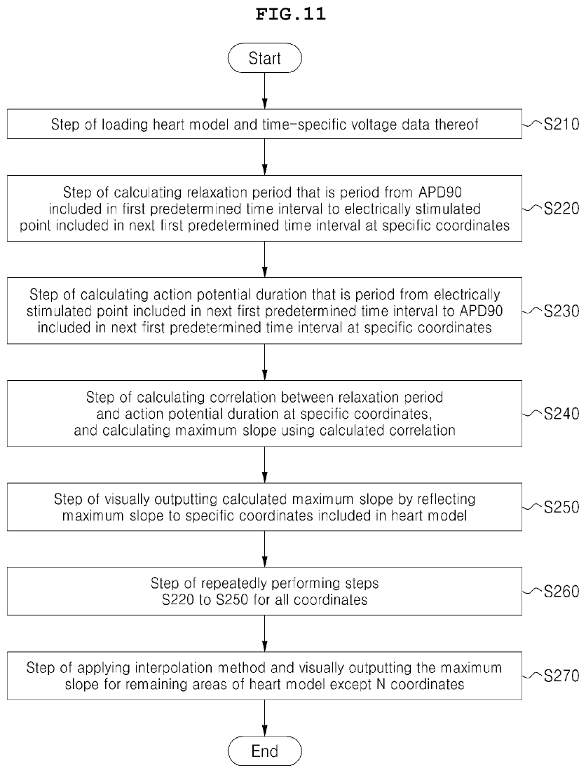

FIG. 10 is a view showing the maximum slope at specific coordinates in color in the atrium model shown in FIG. 3. Since the specific coordinates are a single point, it will be difficult for a user to identify when the point is displayed only in color. Accordingly, as shown in FIG. 11, it is possible to further perform the steps of repeatedly performing steps S220 to S250, after step S250, for all the N coordinates included in the heart model except the specific coordinates (S260), and applying an interpolation method to the maximum slope calculated for the N coordinates included in the heart model, and visually outputting the maximum slope, for the remaining areas of the heart model except the N coordinates included in the heart model (S270).

The above description of steps S220 to S250 is about any specific coordinates among the N coordinates included in the heart model, and when steps S220 to S250 are performed for all N coordinates except the specific coordinates according to step S260, the maximum slope may be visually output for all N coordinates. However, since the N coordinates are N points also in this case, there may be areas that are not visually output between the coordinates, and this can be solved by step S270.

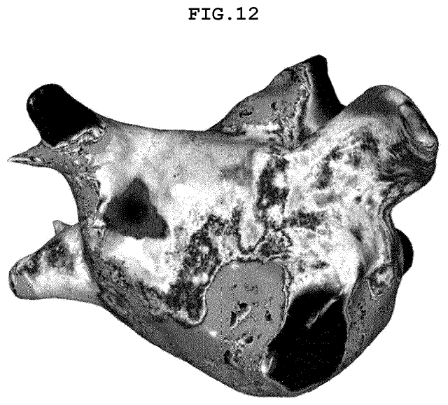

Here, as the interpolation method is to visually output an area to be interpolated on the basis of the things visually output around the area to be interpolated or the maximum slope, the area may be visually output using red, orange, yellow, green, blue, indigo and purple in order of the magnitude of the maximum slope, and a heart model according thereto is shown in FIG. 12.

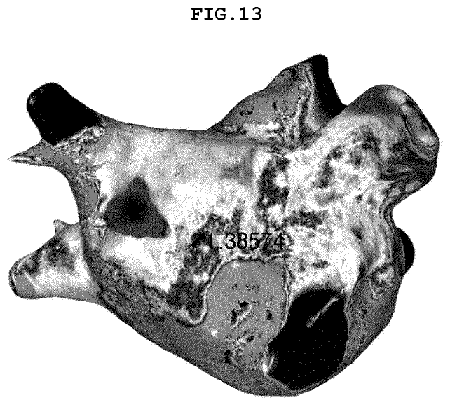

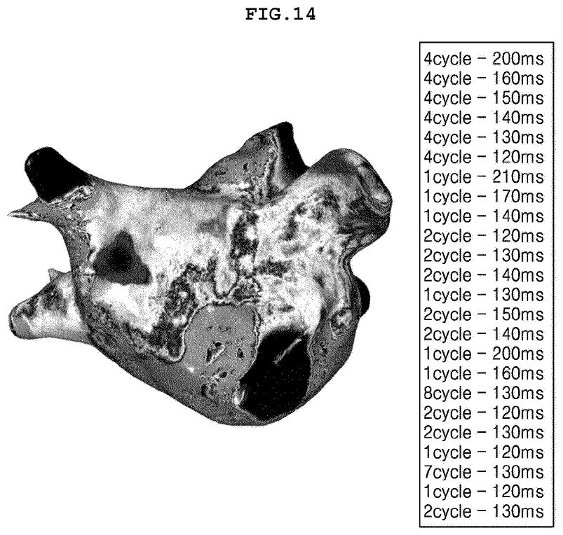

On the other hand, the black area in the left middle of the heart model shown in FIG. 12 means a stimulated area, and when the user selects specific coordinates of the heart model through an input device such as a mouse or the like as shown in FIG. 13, the maximum slope at the corresponding coordinates may be output as a numeric value as described above, or the stimulation cycle of the electrical signal may be output as a numeric value together with the heart model as shown in FIG. 14.

A heart model generation method reflecting action potential duration restitution according to a second embodiment of the present invention has been described above. Since it has been derived from the study that coordinates, at which the magnitude of the maximum slope of the correlation between the relaxation period and the action potential duration is 1 or more, may be regarded as a point where atrial fibrillation occurs or a point where atrial fibrillation is highly likely to occur, a user may accurately detect a point where atrial fibrillation occurs and a point where atrial fibrillation is highly likely to occur before a radiofrequency catheter ablation procedure while confirming a finally output heart model in real-time. In addition, since the time-specific voltage data used in generating the finally output heart model is a result data of a test generally performed for patients of arrhythmia, and the cost is not high, there is an effect of minimizing the economic burdens of the patients.

Meanwhile, the heart model generation method reflecting action potential duration restitution according to a second embodiment of the present invention may be implemented as a computer program stored in a storage medium to be executed by a computer.

Although not described in detail to prevent duplicate description, the computer program stored in a storage medium may also perform the same steps as those of the heart model generation device reflecting action potential duration restitution according to a second embodiment of the present invention described above, and accordingly, the same effect may be derived. For example, in combination with a computing device, the computer program stored in a medium may execute the steps of: (a) loading a heart model including N (N is a natural number equal to or greater than 1) coordinates, and time-specific voltage data including voltage values measured at N coordinates included in the heart model at first predetermined time intervals; (b) calculating a relaxation period that is a time period from a point APD90 showing a voltage value dropped 90% from the highest point of the voltage value included in a first predetermined time interval to an electrically stimulated point included in a next first predetermined time interval at specific coordinates included in the heart model using the loaded time-specific voltage data; (c) calculating an action potential duration that is a time period from an electrically stimulated point included in the next first predetermined time interval to a point APD90 showing a voltage value dropped 90% from the highest point of the voltage value included in the next first predetermined time interval at specific coordinates included in the heart model using the loaded time-specific voltage data; (d) calculating a correlation between the relaxation period and the action potential duration at specific coordinates included in the calculated heart model, and calculating a maximum slope using the calculated correlation; and (e) visually outputting the calculated maximum slope by reflecting the maximum slope to specific coordinates included in the heart model.

Although the embodiments of the present invention have been described above with reference to the accompanying drawings, those skilled in the art may understand that the present invention can be implemented in other specific forms without changing the technical spirit or essential features. Therefore, it should be understood that the embodiments described above are illustrative and not restrictive in all respects.

1. A heart model generation method reflecting action potential duration restitution executed in a heart model generation device reflecting action potential duration restitution, the method comprising the steps of:
(a) loading a heart model including N (N is a natural number equal to or greater than 1) coordinates, and time-specific voltage data including voltage values measured at N coordinates included in the heart model at first predetermined time intervals;
(b) calculating a relaxation period that is a time period from a point APD90 showing a voltage value dropped 90% from the highest point of the voltage value included in a first predetermined time interval to an electrically stimulated point included in a next first predetermined time interval at specific coordinates included in the heart model using the loaded time-specific voltage data;
(c) calculating an action potential duration that is a time period from an electrically stimulated point included in the next first predetermined time interval to a point APD90 showing a voltage value dropped 90% from the highest point of the voltage value included in the next first predetermined time interval at specific coordinates included in the heart model using the loaded time-specific voltage data;
(d) calculating a correlation between the relaxation period and the action potential duration at specific coordinates included in the calculated heart model, and calculating a maximum slope using the calculated correlation; and
(e) visually outputting the calculated maximum slope by reflecting the maximum slope to specific coordinates included in the heart model.

2. The method according to claim 1, wherein the heart model is a three-dimensional atrium model generated for each patient.

3. The method according to claim 1, wherein the N coordinates are 450,000 coordinates.

4. The method according to claim 1, wherein the first predetermined time interval is any one among 1 ms, 2 ms, and 3 ms.

5. The method according to claim 1, wherein the correlation between the relaxation period and the action potential duration at step (d) is calculated through a correlation calculation formula shown below:
y(action potential duration)=y₀−A₁(1−e^{-relaxation period/τ1}) Correlation calculation formula:
where yₒ and A₁ are free-fitting variables, and τ₁ is a time constant.

6. The method according to claim 5, wherein the maximum slope is calculated by differentiating the correlation calculation formula with respect to the relaxation duration.

7. The method according to claim 1, further comprising, after step (e), the step of (f) repeatedly performing steps (b) to (e) for all the N coordinates included in the heart model except the specific coordinates.

8. The method according to claim 7, further comprising, after step (f), the step of (g) applying an interpolation method to the maximum slope calculated for the N coordinates included in the heart model, and visually outputting the maximum slope, for remaining areas of the heart model except the N coordinates included in the heart model.

9. The method according to claim 1, wherein a range of a magnitude of the calculated maximum slope is 0.3 to 2.3, and a visual output of step (e) is outputting the maximum slope in a different color according to the magnitude of the calculated maximum slope.

10. A heart model generation device reflecting action potential duration restitution, the device comprising:
one or more processors;
a network interface;
a memory for loading a computer program executed by the processors; and
a storage for storing large-scale network data and the computer program,
wherein the computer program executes, by the one or more processors 10,
(a) an operation of loading a heart model including N (N is a natural number equal to or greater than 1) coordinates, and time-specific voltage data including voltage values measured at N coordinates included in the heart model at first predetermined time intervals,
(b) an operation of calculating a relaxation period that is a time period from a point APD90 showing a voltage value dropped 90% from the highest point of the voltage value included in a first predetermined time interval to an electrically stimulated point included in a next first predetermined time interval at specific coordinates included in the heart model using the loaded time-specific voltage data,
(c) an operation of calculating an action potential duration that is a time period from an electrically stimulated point included in the next first predetermined time interval to a point APD90 showing a voltage value dropped 90% from the highest point of the voltage value included in the next first predetermined time interval at specific coordinates included in the heart model using the loaded time-specific voltage data,
(d) an operation of calculating a correlation between the relaxation period and the action potential duration at specific coordinates included in the calculated heart model, and calculating a maximum slope using the calculated correlation, and
(e) an operation of visually outputting the calculated maximum slope by reflecting the maximum slope to specific coordinates included in the heart model.

11. A computer program stored in a medium to execute, in combination with a computing device, the steps of:
(a) loading a heart model including N (N is a natural number equal to or greater than 1) coordinates, and time-specific voltage data including voltage values measured at N coordinates included in the heart model at first predetermined time intervals;
(b) calculating a relaxation period that is a time period from a point APD90 showing a voltage value dropped 90% from the highest point of the voltage value included in a first predetermined time interval to an electrically stimulated point included in a next first predetermined time interval at specific coordinates included in the heart model using the loaded time-specific voltage data;
(c) calculating an action potential duration that is a time period from an electrically stimulated point included in the next first predetermined time interval to a point APD90 showing a voltage value dropped 90% from the highest point of the voltage value included in the next first predetermined time interval at specific coordinates included in the heart model using the loaded time-specific voltage data;
(d) calculating a correlation between the relaxation period and the action potential duration at specific coordinates included in the calculated heart model, and calculating a maximum slope using the calculated correlation; and
(e) visually outputting the calculated maximum slope by reflecting the maximum slope to specific coordinates included in the heart model.
